(19) United States
(12) Patent Application Publication
(10) Pub. No.: US 2025/0295929 A1
(43) Pub. Date: Sep. 25, 2025

(54) IMPLANTABLE MEDICAL DEVICE

(71) Applicant: BIOTRONIK SE & Co. KG, Berlin (DE)

(72) Inventor: René FISCHER, Berlin (DE)

(73) Assignee: BIOTRONIK SE & Co. KG, Berlin (DE)

(21) Appl. No.: 18/863,032

(22) PCT Filed: Apr. 26, 2023
(86) PCT No.: PCT/EP2023/060925

§ 371 Date: Nov. 5, 2024

(30) Foreign Application Priority Data
May 19, 2022 (EP) 22174201.8

(51) Int. Cl. A61N 1/39

(52) U.S. Cl. CPC A61N 1/3987; A61N 1/3956

(57) ABSTRACT

An implantable medical device for stimulating a human or animal heart. During operation, the device performs the following steps: a) repeatedly detecting a cardiac electric signal; b) determining a duration of a cardiac interval from the detected cardiac electric signal and calculating a theoretic cardiac rate for each interval from the determined duration; c) determining a percentage of cardiac intervals lying within a first band having a predeterminable first width around a border between first and second cardiac rate zones in which the implantable medical device is operated in first and second operational modes, respectively; and d) if the determined percentage exceeds a predeterminable threshold, i) outputting a notification signal being indicative for a recommended adjustment of the border between the first and second cardiac rate zones, or ii) automatically adjusting the border between the first and second cardiac rate zones.

### CROSS-REFERENCE TO RELATED APPLICATIONS

This application is the United States National Phase under 35 U.S.C. § 371 of PCT International Patent Application No. PCT/EP2023/060925, filed on Apr. 26, 2023, which claims the benefit of European Patent Application No. 22174201.8, filed on May 19, 2022, the disclosures of which are hereby incorporated by reference herein in their entireties.

### TECHNICAL FIELD

The present invention relates to an implantable medical device for stimulating a human or animal heart according to the preamble of claim 1 and to a method for optimizing programming parameters of such an implantable medical device according to the preamble of claim 14.

### BACKGROUND

Most of the programming parameters of an implantable medical device are set during the initial programming of the device by a clinical user. In many cases, standard values proposed by the manufacturer of the device are used for performing this initial programming. Typically in case of (repeated) inadequate decisions made by the implantable medical device, specific parameters are then (iteratively) manually adjusted to the needs of the individual patient to whom the implantable medical device is implanted.

Due to this manual readjustment of the programming parameters of prior art devices, repeated inadequate decisions may be made by the applied algorithms. Only by the active, manual reprogramming, this undesired state can be overturned. The reaction time between the first occurrence of an adverse detection/stimulation scenario until the relieving readjustment is additionally dependent on the presence and the usage behavior of telemetry system (such as a home monitoring system) as well as the experience of the clinical user responsible for the readjustment of the programming parameters.

U.S. Pat. No. 7,769,436 B1 describes an implantable medical device that serves for detecting an abnormal physiological condition within a patient. During operation of the implantable medical device, in particular during an initial learning period, the device collects information representative of a range of variation in the morphologic parameter within the patient. The device then adjusts the detection threshold based on the information representative on the range of variation in the morphologic parameter to improve the detection accuracy.

The present disclosure is directed toward overcoming one or more of the above-mentioned problems, though not necessarily limited to embodiments that do.

### SUMMARY

It is an object of the present invention to provide an implantable medical device, the programming parameters of which can be adjusted during operation of the device according to the physiological needs of the patient carrying the device, wherein the programming parameters do not exclusively influence a detection specificity of the device.

At least this object is achieved with an implantable medical device having the claim elements of claim 1. Such an implantable medical device serves for stimulating a human or animal heart. The device comprises a processor, a memory unit, a stimulation unit and a detection unit. The stimulation unit serves for stimulating a human or animal heart. The detection unit serves for detecting an electric signal of the same heart.

According to an aspect of the present invention, the memory unit comprises a computer-readable program that causes the processor to perform the steps explained in the following when executed on the processor.

In one of the steps, a cardiac electric signal is repeatedly detected with the detection unit. This results in an intracardiac electrogram (IEGM).

In another step, a duration of the cardiac interval is determined from the detected cardiac electric signal. Furthermore, a theoretic cardiac rate is calculated for each of these intervals from the determined duration. Typically, the interval is determined between two identical cardiac events succeeding one another, e.g., between two R wave or between two T waves, resulting in an R-R interval or a T-T interval. If such an interval has a duration of, e.g., 0.5 seconds, a theoretic cardiac rate of 120 bpm results. If the duration is, e.g., only 0.3 seconds, a theoretic cardiac rate of 180 bpm results.

In another method step, the percentage of cardiac intervals lying within a first band is determined. This first band has a predeterminable first width around a border between a first cardiac rate zone and a second cardiac rate zone. Generally, the implantable medical device is programmed such that it is operated in a first operational mode if the theoretic cardiac rate lies within the first cardiac rate zone. Likewise, the device is configured such to be operated in a second operational mode if the theoretic cardiac rate lies within the second cardiac rate zone. Otherwise, if a rhythm with a heart rate near the zone border (e.g., border between cardiac rate zone 1 and 2) is present, a mixture between both operational modes can cause inadequate decisions.

In another method step, one of at least two possible actions is performed by the implantable medical device if the determined percentage exceeds a predeterminable threshold. According to a first possibility, a notification signal is output by the device, the signal being indicative for a recommended adjustment of the border between the first cardiac rate zone and the second cardiac rate zone. This notification signal may result in an alert being provided to a clinical user of the implantable medical device. The clinical user can then decide whether or not to follow the recommended adjustment of the border between the first cardiac rate zone and the second cardiac rate zone. Such a user interaction prior to a final readjustment of the border between the first cardiac rate zone and the second cardiac rate zone may be required according to the marketing authorization stipulations in certain countries. According to the second alternative, the implantable medical device automatically adjusts the border between the first cardiac rate zone and the second cardiac rate zone if the above-mentioned condition is fulfilled. Thus, according to the second alternative, a fully automated adjustment of the border between the first cardiac rate zone and the second cardiac rate zone (i.e., a specific programming parameter of the implantable medical device) is carried out.

The presently described implantable medical device is particularly appropriate to ameliorate a stimulation therapy provided to a patient between implantation of the device (when the device receives its initial programming) until the first occurrence of a cardiac state which requires therapy. The frequency of inadequate decisions made by the device on the basis of an insufficient knowledge on the specific cardiac rates at which pathologic cardiac states occur that require therapy is significantly reduced. This is particularly helpful in case of patients that are preventively provided with the implantable cardiac device, i.e., patients that received the device as primary prophylaxis without having knowledge on an occurrence of any concrete cardiac states of the patient that require therapy or on the symptoms connected to such cardiac states.

In contrast to prior art solutions, the presently described and claimed implantable medical device does not compare morphologic parameters with threshold values. Rather, it extracts timing information from the electric cardiac electric signal and checks whether the chosen programming of the implantable medical device is appropriate for the extracted timing information or rather presents an inappropriate choice of programming parameters. In the latter case, an optimization of the programming parameters, namely, of at least one border between different cardiac rate zones, is performed. A programming of the implantable medical device results that suits the physiologic needs of the patient carrying the implantable medical device.

In an embodiment, the implantable medical device is an implantable pulse generator (IPG), an implantable cardioverter-defibrillator (ICD), or a device for cardiac resynchronization therapy (CRT).

In an embodiment, the adjustment of the border between the first cardiac rate zone and the second cardiac rate zone is recommended or performed such that the border is to be shifted such that the cardiac rate zone is enlarged to which the plurality of cardiac intervals has been assigned to.

In an embodiment, the predeterminable first width lies in a range of from 4% to 15%, in particular from 5% to 14%, in particular from 6% to 13%, in particular from 7% to 12%, in particular from 8% to 11%, in particular from 9% to 10% of the cardiac rate of the border. To give an example, if the border between the first cardiac rate zone and the second cardiac rate zone is set at a cardiac rate of 170 bpm and the first width is set to, e.g., 10%, the first band would have a first width of 17 bpm. Thereby, the first band is typically symmetrically arranged around the border so that the band generally has a width corresponding to ±2% to ±7.5% of the cardiac rate of the border. In the preceding example, the first band has a width of ±5%. According to an embodiment, the predeterminable first width is adjustable by a user.

In an embodiment, the predeterminable threshold lies in a range of from 10% to 75%, in particular from 20% to 70%, in particular of from 30% to 60%, in particular of from 40% to 50%. According to an embodiment, the predeterminable threshold is adjustable by a user.

In an embodiment, the automatic adjusting of the border is limited to a predeterminable optimizing rate per optimization iteration. In doing so, the risk of adjusting the border to non-physiologic values is significantly limited. Furthermore, a limitation of the possible adjustment of the border also reflects the fact that the cardiac rate of the patient may (significantly) vary over time. Then, it would be counter-productive to adjust the border in a single step to a significantly different value than the initial value, whereas the cardiac rate of the patient may change to different values after a short time.

In an embodiment, the predeterminable optimizing rate lies in a range of from 1% to 6%, in particular from 2% to 5%, in particular from 3% to 4% of the cardiac rate of the border. To give an example, if the border is set to a cardiac rate of 170 bpm, a predeterminable optimizing rate of 3% would allow an adjustment of the border by approximately 5 bpm per optimization iteration.

In an embodiment, the maximum possible adjustment of the border is limited to a predeterminable maximum value. In doing so, the risk of adjusting the border in a plurality of optimization iterations to more inappropriate values is significantly reduced.

In an embodiment, the shortest period between two optimization iterations is limited to a predeterminable minimum value. This value could be aligned with the updating interval of the telemonitoring system—typically 24 h. In doing so, the risk of adjusting the border in a plurality of optimization iterations to more inappropriate values is reduced.

In an embodiment, the first operational mode is a standard mode. In the standard mode, the computer-readable program causes the processor not to perform further analysis of the cardiac electric signal other than the steps of determining the duration of the cardiac interval from the detected cardiac electric signal and calculating a theoretic cardiac rate for each interval from the determined duration as well as determining the percentage of cardiac intervals lying within the first band. In this embodiment, the second operational mode is either a monitoring mode or a therapeutic mode. In the monitoring mode, the computer-readable program causes the processor to differentiate between a ventricular tachycardia that requires therapy and a tachycardia having a different origin (such as a supraventricular tachycardia) and not requiring therapy. In the therapeutic mode, the computer-readable program causes the processor to emit at least one stimulation pulse (e.g., a series of pulses to induce a cardioversion and defibrillation). Optionally, the therapeutic mode also enables a differentiation between a ventricular tachycardia requiring therapy and a tachycardia having a different origin (such as a supraventricular tachycardia) and not requiring therapy. A therapeutic mode with a full detection logic, i.e., with the possibility to differentiate between a ventricular tachycardia requiring therapy and a tachycardia not requiring therapy can also be denoted as ventricular tachycardia mode. A therapeutic mode that does not allow such a differentiation, but always enables the emission of at least one stimulation pulse can also be referred to as ventricular fibrillation mode. It can be in particular applied in case of high (theoretic) cardiac rates of the patient in which the probability of ventricular fibrillation is much higher than the probability of a ventricular tachycardia that may not require therapy.

In an embodiment, the computer-readable program causes the processor to perform the step of outputting a notification signal or automatically adjusting the border between the first cardiac rate zone and the second cardiac rate zone if the determined percentage exceeds the predeterminable threshold upon detection of a termination of a tachycardic episode. Thus, the reprogramming of the border between the first cardiac rate zone and the second cardiac rate zone is not performed during the tachycardic episode, but rather after the tachycardic episode has been terminated. This ensures a reliable therapy of the present tachycardic episode, but still enables an evaluation and adjustment of the therapeutic efficacy of the implantable medical device with respect to the individual physiologic needs of the patient for future tachycardic episodes.

In an embodiment, the computer-readable program causes the processor to perform the step of outputting a notification signal or automatically adjusting the border between the first cardiac rate zone and the second cardiac rate zone if the determined percentage exceeds the predeterminable threshold in regular intervals, such as once a day, once in 2 days, once in a week, once in 2 weeks, once in a month, once in 2 months, once in 3 months, once in 6 months, once in a year, or in any interval that lies in a range which can be built up from the precedingly mentioned intervals, e.g., falling in a range between once in a day and once in a year. By performing the step of outputting the notification signal or automatically adjusting the border in such regular intervals, it is possible to retrospectively evaluate the measured cardiac rate of a specific preceding interval and to continuously optimize the functional parameters of the implantable medical device with respect to the physiological needs of the patient.

In an embodiment, the computer-readable program causes the processor to perform an additional step for checking a stability of the theoretic cardiac rate. In this context, the theoretic cardiac rate is classified as stable if at least 20%, 30%, 40%, 50%, 60%, 70%, 80%, or 90% of the analyzed intervals have a duration lying within a second band having a predeterminable second width around an average duration of a predeterminable number of preceding intervals. By such an additional stability check, it is ensured that the adjustments of the border between the first cardiac rate zone and the second cardiac rate zone is not performed on the basis of an arhythmic cardiac rate that would not reflect the real cardiac activity of the patient carrying the implantable medical device. Expressed in other words, by checking the stability of the determined (theoretic) cardiac rate, an unintentional compliance with the condition to be fulfilled in order to optimize the border between the first cardiac rate zone and the second cardiac rate zone or to generate a notification signal on an recommended optimization of this border is avoided.

In an embodiment, the predeterminable second width lies in a range of from 2% to 60%, in particular from 5% to 50%, in particular from 10% to 40%, in particular from 20% to 30% of the average duration of the predeterminable number of preceding intervals. Typically, the second width is symmetrically arranged around the average duration of the predeterminable a number of preceding intervals so that a width of 40% corresponds to a band of ±20% around the average duration of the predeterminable number of preceding intervals.

In an embodiment, the predeterminable number of preceding intervals lies in a range of from 2 to 20, in particular from 3 to 19, in particular from 4 to 18, in particular from 5 to 17, in particular from 6 to 16, in particular from 7 to 15, in particular from 8 to 14, in particular from 9 to 13, in particular from 10 to 12. A particularly appropriate predeterminable number of preceding intervals lies in a range of from 2 to 5, e.g., at or around 3. Then, only a rather short preceding section of the cardiac rhythm would be used as reference for determining the stability of the cardiac rate of the patient.

In an embodiment, the computer-readable program causes the processor to additionally perform a morphologic analysis of the cardiac electric signal over time and to perform the step of outputting a notification signal or automatically adjusting the border between the first cardiac rate zone and the second cardiac rate zone only if a predeterminable morphologic criterion is also fulfilled. In this embodiment, the step of outputting the notification signal or automatically adjusting the border thus depends on two conditions, namely, on an exceedance of the predeterminable threshold by the determined percentage and on a fulfillment of the predeterminable morphologic criterion.

In an embodiment, the predeterminable morphologic criterion is indicative for a sinusoidal rhythm of the cardiac electric signal. A peak-to-peak amplitude, a normalized wave difference vector, and/or a normalized area under a signal are particularly appropriate morphologic criteria indicative for a sinusoidal rhythm. It is generally known, how to evaluate such morphologic criteria to classify a cardiac rhythm as sinusoidal or arrhythmic. If a sinusoidal rhythm is given, there is a physiologic confirmation that an adjustment of the border corresponds to the physiologic needs of the patient. If no such sinusoidal rhythm of the cardiac electric signal is given, an adjustment of the border should not be performed automatically since a proper evaluation of the cardiac signal cannot be automatically performed in a reliable way. Rather, a warning signal would be generated in such a case by the implantable medical device to draw the attention of the clinical user of the implantable medical device to the arrhythmic cardiac rhythm of the patient with the concomitant apparent need to adjust the border to properly address the physiologic needs of the patient.

In an aspect, the present invention relates to a method for optimizing a programming parameter of an implantable medical device according to the preceding explanations. This method comprises the steps explained in the following:

In one of the steps, a cardiac electric signal is repeatedly detected with a detection unit of the implantable medical device. This results in an intracardiac electrogram (IEGM).

In another step, a duration of a cardiac interval is determined from the detected cardiac electric signal. Furthermore, a theoretic cardiac rate is calculated for each of these intervals from the determined duration.

In another method step, the percentage of cardiac intervals lying within a first band is determined. This first band has a predeterminable first width around a border between a first cardiac rate zone and a second cardiac rate zone. Generally, the implantable medical device is programmed such that it is operated in a first operational mode if the theoretic cardiac rate lies within the first cardiac rate zone. Likewise, the device is configured such to be operated in a second operational mode if the theoretic cardiac rate lies within the second cardiac rate zone.

In another method step, one of at least two possible actions is performed if the determined percentage exceeds a predeterminable threshold. According to the first alternative, a notification signal is output by the device, the signal being indicative for a recommended adjustment of the border between the first cardiac rate zone and the second cardiac rate zone. This notification signal may result in an alert being provided to a clinical user of the implantable medical device. The clinical user can then decide whether or not to follow the recommended adjustment of the border between the first cardiac rate zone and the second cardiac rate zone. According to the second alternative, the implantable medical device automatically adjusts the border between the first cardiac rate zone and the second cardiac rate zone if the above-mentioned condition is fulfilled. Thus, according to the second alternative, a fully automated adjustment of the border between the first cardiac rate zone and the second cardiac rate zone (i.e., a specific programming parameter of the implantable medical device) is carried out.

All embodiments of the implantable medical device can be combined in any desired manner and can be transferred either individually or in any arbitrary combination to the described method. Likewise, all embodiments of the described method can be combined in any desired way and can be transferred either individually or in any arbitrary combination to the described implantable medical device.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be explained in the following making reference to exemplary embodiments and accompanying Figures. In the Figures:

FIG. 1 shows a schematic depiction of possible cardiac rate zones defining different operational modes of an implantable medical device;

FIG. 2 schematically illustrates a borderline cardiac rhythm;

FIG. 3 schematically illustrates possible optimizations of a border between a first cardiac rate zone and a second cardiac rate zone in case of a borderline cardiac rhythm; and

FIG. 4 schematically illustrates an iterated optimization process of the border between a first cardiac rate zone and a second cardiac rate zone.

### DETAILED DESCRIPTION

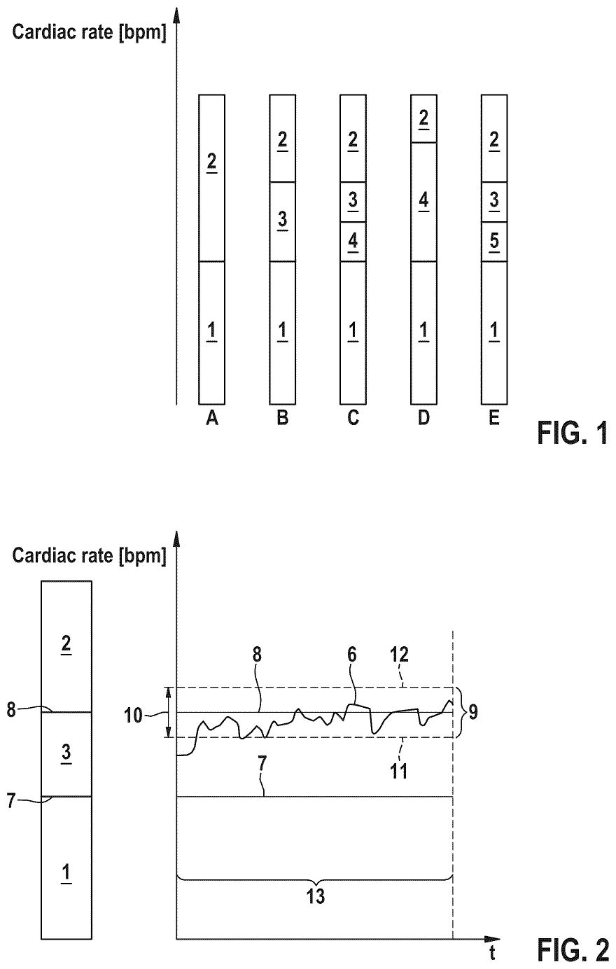

FIG. 1 schematically illustrates a possible programming of different cardiac rate zones defining different operational modes of an implantable medical device for stimulating a human or animal heart, e.g., of an implantable cardioverter-defibrillator (ICD). According to programming variant A, a standard zone (also referred to as non-tachycardic zone) 1 and a ventricular fibrillation zone 2 are defined. If a cardiac rate is detected lying within the standard zone 1, the implantable medical device is operated according to a first operational mode in which no discrimination between ventricular tachycardia and ventricular fibrillation is made. Rather, only the cardiac rate is determined. In addition, during this standard operational mode (also referred to as non-tachycardic mode), no therapy delivery (by emitting a stimulation pulse by a stimulation unit of the implantable medical device) is necessary nor possible. In contrast, if a cardiac rate lying within the ventricular fibrillation zone 2 is detected, the implantable medical device is operated in a ventricular fibrillation mode. In this ventricular fibrillation mode, a therapy will be delivered by emitting stimulation pulses in order to achieve a defibrillation and cardioversion of the heart back to a normal state.

According to programming variant B, a standard zone 1 and a ventricular fibrillation zone 2 as well as a first ventricular tachycardia zone 3 are defined. The operational modes of the implantable medical device applied if a cardiac rate is detected lying within the standard zone 1 or the ventricular fibrillation zone 2 are the same as explained with respect to programming variant A. However, if the cardiac rate falls within the first ventricular tachycardia zone 3, the implantable medical device is operated in a first ventricular tachycardia operational mode. In this mode, a discrimination between ventricular tachycardia requiring therapy and a tachycardia having another origin and not requiring therapy is made. Furthermore, a therapy is delivered in case that a ventricular tachycardia requiring therapy has been detected. Thus, in contrast to the ventricular fibrillation mode which is acquired in case of the cardiac rate falling within the ventricular fibrillation zone 2 and in which a therapy is always delivered, such therapy delivery is dependent on the fulfilment of a further condition in case of operating the implantable medical device in the first ventricular tachycardia operational mode that is acquired in case of the cardiac rate falling within the first ventricular tachycardia zone 3.

In the programming variant C, four operational modes of the implantable medical device and thus four cardiac rate zones are defined, namely, a standard zone 1, a ventricular fibrillation zone 2, a first ventricular tachycardia zone 3 lying below the ventricular fibrillation zone 2, and a ventricular tachycardia monitoring zone 4 lying between the first ventricular tachycardia zone 3 and the standard zone 1. The operational modes of the implantable medical device performed if the cardiac rate falls within the standard zone 1, the ventricular fibrillation zone 2 or the first ventricular tachycardia zone 3 are the same as explained with respect to variants A and B. If the cardiac rate falls within the ventricular tachycardia monitoring zone 4, the implantable medical device is operated according to a ventricular tachycardia monitoring operational mode. In this ventricular tachycardia monitoring operational mode, a full discrimination is made between a ventricular tachycardia requiring therapy and a tachycardia having a different origin and not requiring therapy. However, in this operational mode, the implantable medical device cannot deliver a therapy. Rather, such therapy delivery will only be possible in that operational modes that are activated if the cardiac rate falls within the first ventricular tachycardia zone 3 or the ventricular fibrillation zone 2.

According to the programming variant D, three cardiac rate zones, namely, a standard zone 1, a ventricular fibrillation zone 2, and ventricular tachycardia monitoring zone 4 lying between the standard zone 1 and the ventricular fibrillation zone 2 are defined. Consequently, the implantable medical device can be operated according to three operational modes having the functionality explained with respect to the preceding variants A to C.

According to programming variant E, four cardiac rate zones are provided, namely, a standard zone 1, a ventricular fibrillation zone 2, a first ventricular tachycardia zone 3 lying below the ventricular fibrillation zone 2, and a second ventricular tachycardia zone 5 lying between the first ventricular tachycardia zone 3 and the standard zone 1. The operational modes activated if a cardiac rate falls within the second ventricular tachycardia zone 5 is similar to the operational mode that is activated by a cardiac rate falling within the first ventricular tachycardia zone 3. Thus, also in the operational mode activated by the second ventricular tachycardia zone 5, a discrimination between a ventricular tachycardia and a tachycardia having a different origin is made. However, the applied algorithms in the first ventricular tachycardia operational mode and in the second ventricular tachycardia operational mode are slightly different. In any case, it is possible to deliver therapy also in the second ventricular tachycardia operational mode activated if the cardiac rate falls within the second ventricular tachycardia zone 5.

FIG. 2 schematically illustrates how a borderline cardiac rhythm is defined and identified. For this purpose, FIG. 2 shows the theoretic cardiac rate 6 of a patient that is calculated for each RR-interval of the patient's cardiac rhythm detected with an implantable cardioverter-defibrillator (ICD) serving as implantable medical device. The ICD can be operated in three operational modes. It is operated in a first operational mode or standard mode, if the detected cardiac rate falls within the standard cardiac rate zone 1. In this and in all following Figures, similar elements will be denoted with the same numeral references as in the other Figures. The ICD is operated in ventricular fibrillation mode if the detected cardiac rate falls within the ventricular fibrillation zone 2. The ICD is operated in a first ventricular tachycardia operational mode if the cardiac rate falls within the first ventricular tachycardia zone 3. There is a first border 7 defined between the standard zone 1 and the first ventricular tachycardia zone 3. Furthermore, a second border 8 is defined between the first ventricular tachycardia zone 3 and the ventricular fibrillation zone 2. The first border 7 and the second border 8 are also illustrated in the diagram of the temporal course of the cardiac rate. There is a first band 9 defined around the second border 8. The first band 9 has a width 10 ranging from a lower threshold 11, which is arranged at −5% of the cardiac rate of the second border 8, and an upper threshold 12, which is defined at +5% of the cardiac rate of the second border 8. Consequently, the width 10 of the first band 9 amounts to 10% of the cardiac rate of the second border 8.

The high theoretic cardiac rate 6 remains close to the second border 8 during a duration 13 of a ventricular tachycardia episode. Upon termination of this ventricular tachycardia episode, the theoretic cardiac rate drops to lower values.

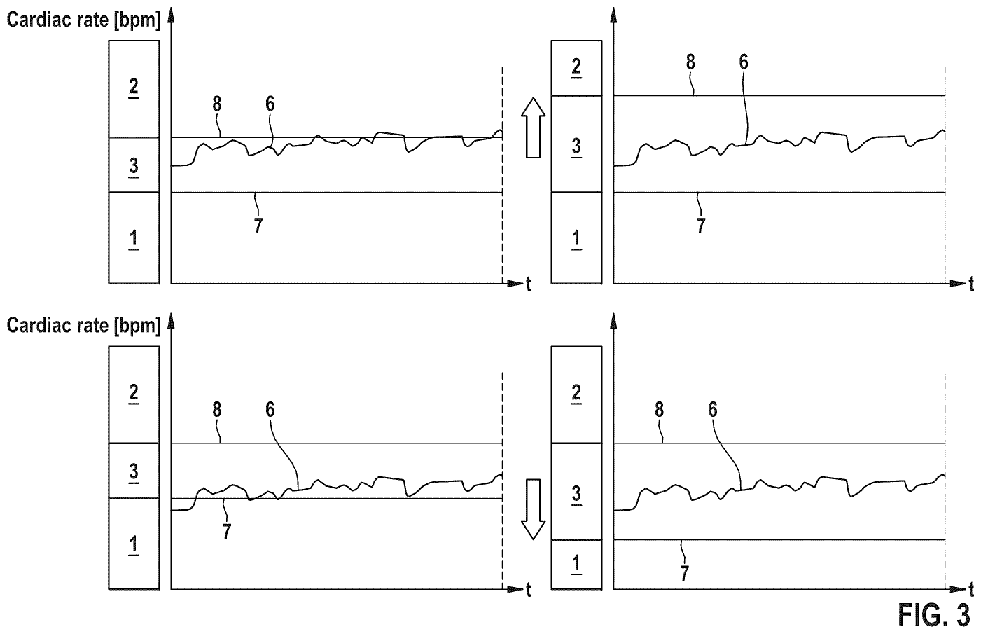

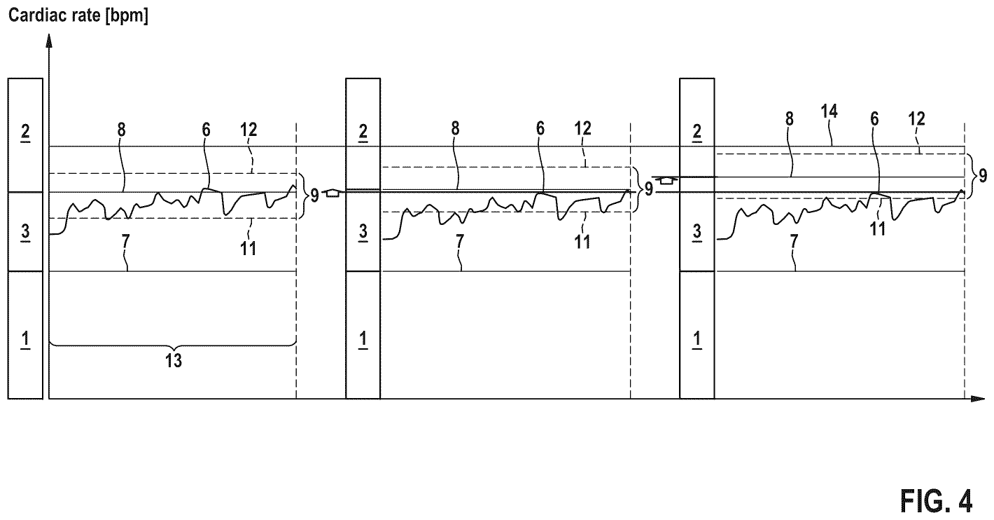

The patient's theoretic cardiac rate 6 is very close to the second border 8 between the ventricular fibrillation zone 2 and the first ventricular tachycardia zone 3. In fact, more than 30% of all values of the theoretic cardiac rate 6 lie within the first band 9. Consequently, it is difficult for the underlying algorithm to make a decision whether the operational mode assigned to the ventricular fibrillation zone 2 or the operational mode assigned to the first ventricular tachycardia zone 3 is to be applied for the ICD. Expressed in other words, the theoretic cardiac rate 6 represents a borderline cardiac rhythm for which the programming parameters of the ICD are not optimally adjusted. However, the ICD is able to solve at least this problem, as will be illustrated in more detail with respect to FIGS. 3 and 4.

In the upper panel of FIG. 3, a similar situation like in FIG. 2 is illustrated. Here, the theoretic cardiac rate 6 of the patient is close to the second border 8. Since more than 30% of the intervals lie within the band 9 defined around the second border 8 (not depicted in FIG. 3, cf. FIG. 2 for more details), the second border 8 is shifted upwards. As a result, the first ventricular tachycardia zone 3 is enlarged, whereas the ventricular fibrillation zone 2 is made smaller. Due to this shift of the second border 8, the theoretic cardiac rate 6 now clearly lies within the first ventricular tachycardia zone 3 so that the ICD can be safely operated in the first ventricular tachycardia operating mode. Due to the shift of the second border 8 to a higher cardiac rate, the operational parameter of the ICD that have turned out to be inappropriate for the specific cardiac rate of the patient has been adjusted to better suit the physiologic needs of the patient. Due to this shift of the second border 8, the theoretic cardiac rate 6 is no longer a borderline cardiac rhythm, but rather represents a cardiac rhythm clearly assigned to the first ventricular tachycardia zone 3.

The second border 8 is shifted upwards since most of the values of the theoretic cardiac rate 6 lie within the first ventricular tachycardia zone 3 (even though they are located closely to the second border 8 between the first ventricular tachycardia zone 3 and the ventricular fibrillation zone 2).

In the lower panel of FIG. 3, a situation is illustrated, in which the theoretic cardiac rate 6 is close to the first border 7. Also here, more than 30% of the values of the theoretic cardiac rate 6 lie within a band defined around the first border 7. For illustration purposes, this band is not depicted in FIG. 3. However, it is made up in the same way as the first band 9 illustrated in FIG. 2.

Since more than 30% of the theoretic cardiac rate lie within the band around the first border 7, and since most of the values of the theoretic cardiac rate 6 lie within the first ventricular tachycardia zone 3, the first border 7 is shifted downwards to a lower cardiac rate, thus enlarging the first ventricular tachycardia zone 3 and making the standard zone 1 smaller. The ventricular fibrillation zone 2 remains unaffected from this shift of the first border 7. As a result, the theoretic cardiac rate 6 clearly lies within the enlarged first ventricular tachycardia zone 3 after the shift of the first border 7. Consequently, the ICD can be safely operated in the first ventricular tachycardia operational mode assigned to the first ventricular tachycardia zone 3.

FIG. 4 schematically illustrates a border optimization process that is similar to the process illustrated in FIG. 3. However, the optimization process is performed in two succeeding steps. Initially (left diagram of FIG. 4), the theoretic cardiac rate 6 lies close to the second border 8 within the first band 9 arranged around the second border 8. Consequently, the second border 8 is shifted upwards to enlarge the first ventricular tachycardia zone 3. However, the maximum optimization is limited to 3% of the cardiac rate assigned to the second border 8. The situation after this shift of the second border 8 by 3% is illustrated in the middle diagram of FIG. 4. More than 30% of the values of the theoretic cardiac rate 6 still lie within the first band 9 arranged around the shifted second border 8. Consequently, the second border 8 is once again shifted upwards in a second optimization step. This shift also corresponds to 3% of the current cardiac rate value assigned to the second border 8. Due to this shift, the first ventricular tachycardia zone 3 is further enlarged, and the ventricular fibrillation zone 2 is once again made smaller (right diagram of FIG. 4). As a result, less than 30% of the values of the theoretic cardiac rate 6 lie within the first band 9 arranged around the shifted second border 8. Thus, the theoretic cardiac rate 6 has been redefined from a borderline cardiac rhythm to a cardiac rhythm clearly lying within the enlarged first ventricular tachycardia zone 3 due to the iterated shifting of the second border 8 upwards. Due to this optimization of the definition of the second border 8, a safe operation of the ICD with respect to the detected cardiac rhythm of the patient is made possible.

It should be noted that the optimization process is performed after the end of the duration 13 of a ventricular tachycardia episode. Thus, the optimization process is generally done retrospectively with respect to already calculated cardiac rate values. The optimized parameters (i.e., the optimized definition of the second border 8) are then applied for the future operation of the ICD.

To avoid an unlimited shift of the second border 8, a maximum allowable value 14 is defined which may not be exceeded by the second border 8. This ensures that the ICD will be operated in the ventricular fibrillation mode if the theoretic cardiac rate further increases.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Implantable medical device for stimulating a human or animal heart, comprising a processor, a memory unit, a stimulation unit configured to stimulate a human or animal heart, a detection unit configured to detect an electric signal of the same heart, wherein
in that the memory unit comprises a computer-readable program that causes the processor to perform the following steps when executed on the processor:
a) repeatedly detecting, with the detection unit, a cardiac electric signal;
b) determining a duration of a cardiac interval from the detected cardiac electric signal and calculating a theoretic cardiac rate for each interval from the determined duration;
c) determining a percentage of cardiac intervals lying within a first band having a predeterminable first width around a border between a first cardiac rate zone which the implantable medical device is operated in a first operational mode and a second cardiac rate zone in which the implantable medical device is operated in a second operational mode; and
d) if the determined percentage exceeds a predeterminable threshold, i) outputting a notification signal being indicative for a recommended adjustment of the border between the first cardiac rate zone and the second cardiac rate zone or ii) automatically adjusting the border between the first cardiac rate zone and the second cardiac rate zone.

2. Implantable medical device according to claim 1, wherein the predeterminable first width lies in a range of from 4% to 15% of a cardiac rate of the border.

3. Implantable medical device according to claim 1, wherein the predeterminable threshold lies in a range of from 10% to 75%.

4. Implantable medical device according to claim 1, characterized in that the automatic adjusting of the border is limited to a predeterminable optimizing rate per optimization iteration.

5. Implantable medical device according to claim 4, wherein the predeterminable optimizing rate lies in a range of from 1% to 6% of a cardiac rate of the border.

6. Implantable medical device according to claims 1, wherein the first operational mode is a standard mode, in which the computer-readable program causes the processor not to perform further analyses of the cardiac electric signal exceeding steps b) and c), and in that the second operational mode is i) a monitoring mode, in which the computer-readable program causes the processor to differentiate between a ventricular tachycardia that requires therapy and a tachycardia having a different origin and not requiring therapy, or ii) a therapeutic mode, in which the computer-readable program causes the processor to emit at least one stimulation pulse and optionally to differentiate between a ventricular tachycardia that requires therapy and a tachycardia having a different origin and not requiring therapy.

7. Implantable medical device according to claim 1, wherein the computer-readable program causes the processor to perform step d) upon detection of a termination of a tachycardic episode.

8. Implantable medical device according to claim 1, wherein the computer-readable program causes the processor to perform step d) in regular intervals.

9. Implantable medical device according to claim 1, wherein the computer-readable program causes the processor to perform an additional step for checking a stability of the theoretic cardiac rate, wherein the theoretic cardiac rate is classified as stable if at least 90% of the analyzed intervals have a duration lying within a second band having a predeterminable second width around an average duration of a predeterminable number of preceding intervals.

10. Implantable medical device according to claim 9, wherein the predeterminable second width lies in a range of from 2% to 60% of the average duration of the predeterminable number of preceding intervals.

11. Implantable medical device according to claim 9, wherein the predeterminable number of preceding intervals lies in a range of from 2 to 20.

12. Implantable medical device according to claim 1, wherein the computer-readable program causes the processor to additionally perform a morphologic analysis of the cardiac electric signal over time and to perform step d) only if a predeterminable morphologic criterion is fulfilled.

13. Implantable medical device according to claim 12, wherein the predeterminable morphologic criterion is indicative for a sinusoidal rhythm of the cardiac electric signal.

14. Method for optimizing a programming parameter of an implantable medical device according to claim 1,
wherein the method comprises the following steps:
a) repeatedly detecting, with a detection unit of the implantable medical device, a cardiac electric signal of a patient;
b) determining a duration of a cardiac interval from the detected cardiac electric signal and calculating a theoretic cardiac rate for each interval from the determined duration;
c) determining a percentage of cardiac intervals lying within a first band having a predeterminable first width around a border between a first cardiac rate zone in which the implantable medical device is operated in a first operational mode and a second cardiac rate zone in which the implantable medical device is operated in a second operational mode; and
d) if the determined percentage exceeds a predeterminable threshold, i) outputting a notification signal being indicative for a recommended adjustment of the border between the first cardiac rate zone and the second cardiac rate zone or ii) automatically adjusting the border -between the first cardiac rate zone and the second cardiac rate zone.
